SEQUENCE LISTING (2 sequences)

SEQ ID NO: 1 - DNA, 24 bp, artificial sequence
  Primer ECFP_F used to amplify ECFP gene from plasmid pECFP-1.
gataaggatccgatggtgagcaag

SEQ ID NO: 2 - DNA, 24 bp, artificial sequence
  Primer ECFP_R used to amplify ECFP gene from plasmid pECFP-1.
ttcgaattccttgtacagctcgtc

(19) United States
(12) Patent Application Publication
(10) Pub. No.: US 2003/0068611 A1
(43) Pub. Date: Apr. 10, 2003

(54) Methods for increasing microbial metabolic efficiency through regulation of oxidative stress

(76) Inventors: Robert A. Larossa, Wilmington, DE (US); Ming Zheng, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/098,818

(22) Filed: Mar. 15, 2002

Related U.S. Application Data
(60) Provisional application No. 60/277,501, filed on Mar. 21, 2001.

(51) Int. Cl. C12Q 1/00

(52) U.S. Cl. 435/4

(57) ABSTRACT

Up-regulation of the genetic machinery that regulates the oxidative stress response has been found to increase microbial cell tolerance to toxic substances and to increase the metabolic efficiency of native and recombinant enzymatic pathways, resulting in higher end product yields.

This application claims the benefit of U.S. Provisional Application No. 60/277,501, filed Mar. 21, 2001.

### FIELD OF THE INVENTION

The invention relates to the field of molecular biology and microbiology. More specifically, methods for increasing the metabolic efficiency of cultured microorganisms through the regulation of globally regulated stress response, and particularly the cellular oxidative stress response.

### BACKGROUND OF THE INVENTION

Increasing metabolic efficiency of a production host is one of the constant challenges facing the industrial microbiologist today. Regulation of nutrients and fermentation conditions such as oxygen, nitrogen, inorganic salts, carbon substrates, and end product provides some ability to optimize production to some extent. Additionally, enhancement of fermentative yield has been attempted through addition of growth factors.

For example, Fung has shown that the fermentation process could be improved by the addition of oxygen reactive enzymes that are known to be oxygen reducing to produce anaerobic conditions (U.S. Pat. No. 5,486,367). The addition of these enzymes enhanced the growth of microorganisms in the fermentation system resulting in reduced time to log phase.

However, manipulation of bioprocess conditions to enhance product yield or reduce fermentation time is an imprecise art and such solutions are often only applicable to specific processes. A general method for the enhancement of the product yield of an enzymatic pathway is needed.

It has long been known that up-regulation of genes that are responsive to oxidative stress increases a microbial cell's tolerance to a variety of compounds that produce reactive oxygen species. All organisms that use molecular oxygen must defend themselves from the toxic byproducts of oxygen metabolism. During respiration, reactive species such as hydrogen peroxide, superoxide anion, singlet oxygen, and the hydroxyl radical can be generated in addition to the complete four electron reduction of molecular oxygen to water. These reactive oxygen species can oxidize membrane fatty acids initiating lipid peroxidation, oxidize proteins and damage DNA. Enteric bacteria have several enzymes that may help protect the cells from oxidative damage including superoxide dismutase, catalase (Fridovich, I., Science 201: 875-880, (1978)), exonuclease III (Demple et al., J. Bacteriol. 153: 1079-1083 (1983)), and recA protein (Carlsson and Carpenter, J. Bacterol. 142:319-321, (1980)). When Salmonella typhimurium or Escherichia coli were treated with hydrogen peroxide, catalase activity in cells was induced (Finn and Condon, J. Bacteriol. 123: 570-579 (1975); Richter and Loewen, Biochem. Biophys. Res. Comm. 100:1039-1046 (1981)). Similarly, a treatment of Salmonella typhimurium or Escherichia coli cells with low dose of hydrogen peroxide increased resistance to subsequent lethal dose of hydrogen peroxide and induced the synthesis of 30 plus proteins. A subset of these proteins are encoded by genes regulated by the transcriptional regulator OxyR, including katG (hydroperoxidase), ahpCF (alkylhydroperoxide reductase), oxyS (a regulatory RNA), dps (a non-specific DNA binding protein), fur (ferric uptake regulation), gorA (glutathione reductase) and grxA (glutaredoxin) (Zheng and Storz, Biochem. Pharm., 59:1-6 (2000)). It has been found that strains with an oxyR deletion are unable to induce this regulon and are subsequently hypersensitive to hydrogen peroxide. Strains carrying the dominant mutation oxyR1 in Salmonella typhmurium and oxyR2 in Escherichia coli are resistant to hydrogen peroxide and constitutively overproduce the oxyR-regulated proteins (Christman et al., Cell 41:753-762 (1985)). Furthermore, it has been shown that a dominant missense mutation of the oxyR gene resulted in the overproduction of OxyR-regulated proteins in the absence of oxidative stress (Christman et al., Proc. Natl. Acad. Sci. USA, 86:3484-3488 (1989)). Mutations in OxyR-regulated ahpC gene resulted in higher activity of the enzyme and the mutant strain was resistant to the hydrophobic solvent tetralin (Ferrante et al., Proc. Natl. Acad. Sci. USA, 92:7617-7621 (1995)). Another E. coli transcription factor, SoxR, activates a single gene SoxS in response to superoxide-generating agents and to nitric oxide. The elevated level of SoxS protein leads to increased expression of several genes, including sodA (superoxide dismutase),nfo (endonuclease IV), fpr(ferredoxin/flavodoxin reductase), fldA (flavadoxin) etc. (Zheng and Storz, Biochem. Pharm. 59-1-6 (2000), Storz, C. and Hengge-Aronis, R., Bacterial Stress Responses, American Society for Microbiology Press, Washington, D.C., pp. 47-59, (2000)). In addition to protecting against oxidative damage, SoxRS regulon confers resistance to antibiotics, organic solvents, and heavy metals.

Transcriptional regulators responsive to oxidative stress have been identified in a wide variety of bacterial sp. OxyR has been identified in Mycobacterium, Brucella, Pasteurella, Bacteroides, Erwinia, Streptomyces, Haemophilus, Acinetobacter, Escherichia, Salmonella, Xanthomonas, and Bacillus. SoxRS has been identified in Erwinia, Escherichia, Streptomyces, Pseudomonas, Vibrio, Chromobacterium, Mycobacterium, Arthrobacter, and Salmonella.

Although it is well established that OxyR and SoxRS regulons convey resistance to certain peroxide and superoxide generating substances, no link has been made between this characteristic and metabolic efficiency of the cultured strain. The problem to be solved is to provide a facile, genetically based method of increasing metabolic efficiency of microbial production hosts in order to obtain higher product yields. Applicants have solved the stated problem by providing a method that involves the up-regulation of genes involved in the globally regulated stress response of the cell, and particularly the oxidative stress response, which confers various benefits on the cell. These benefits include increased metabolic efficiency of native and foreign enzymatic pathways, increased expression of recombinant proteins, increased growth rate, and increased resistance to substance toxicity.

### SUMMARY OF THE INVENTION

The invention provides a method for increasing the metabolic efficiency of a microbial enzymatic pathway of interest comprising:

a) providing a microorganism having:

i) at least one highly expressed globally regulated stress responsive gene;

ii) a metabolic enzymatic pathway of interest; and

b) growing the microorganism of (a) for a time sufficient to produce an end-product from the microbial enzymatic pathway of interest.

In a preferred embodiment the highly expressed stress responsive gene is a gene responsive to oxidative stress such as the presence of peroxides or superoxides.

In one embodiment the metabolic efficiency is increased as a result of increased resistance to a chemical substance which may be a solvent or other toxic organic molecule.

In a preferred embodiment the microorganism is an enteric bacteria having a mutation in either or both the oxyR or soxRS oxidative stress genes that results in constitutive expression of those genes.

In another embodiment the metabolic enzymatic pathway of interest results in the over-expression of a protein.

In another preferred embodiment the metabolic enzymatic pathway of interest produces 1,3-propanediol as an end product.

### SEQUENCE DESCRIPTIONS

The following sequences comply with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is the ECFP_F primer

SEQ ID NO:2 is the ECFP_R primer

### DETAILED DESCRIPTION OF THE INVENTION

The present invention describes methods for increasing the metabolic efficiency of a microbial production host harboring an enzymatic pathway of interest. The method involves the up-regulation of genes involved in the globally regulated stress response, and particularly the oxyR and soxRS genes, responsive to oxidative stress. Up-regulation of these genes has been found to increase product yield of native and foreign or introduced enzymatic pathways as well as increasing the host cells tolerance to various toxic by-products of fermentation.

The method has broad applicability in the area of industrial microbiology and in the enhancement of microbial based bio-processes.

In this disclosure, a number of terms and abbreviations are used. The following definitions are provided.

The term “metabolic efficiency” refers to the rate at which the metabolic processes of a cell operate. As the metabolic efficiency of a cell or cellular processes or pathways increase the end-product of those processes or pathways is produced in higher yields.

The term “microbial production host” refers to a microbial cell that comprises an engineered metabolic pathway and which is useful for the production of some product.

The term “stress” or “environmental stress” refers to the condition produced in a cell as the result of exposure to an environmental insult, or over-production of endogenous or exogenous chemicals.

The term “insult” or “environmental insult” refers to any substance or environmental change that results in an alteration of normal cellular metabolism in a bacterial cell or population of cells. Environmental insults may include, but are not limited to, chemicals, environmental pollutants, heavy metals, changes in temperature, changes in pH as well as agents producing oxidative damage, DNA damage, anaerobiosis, changes in nitrate availability or pathogenesis.

The term “toxic organic molecule” refers to any carbon containing material that may be used by a cell as a carbon source but is toxic to that cell.

The term “globally regulated stress response” refers to the cellular response that is genetically regulated via a central genetic system resulting in the induction of either detectable levels of stress proteins or in a state more tolerant to exposure to another insult or an increased dose of the environmental insult.

The term “stress protein” refers to any protein induced as a result of environmental stress or by the presence of an environmental insult. Typical stress proteins include, but are not limited to those encoded by the Escherichia coli genes groEL, groES, dnak, dnaJ, grpE, lon, lysU, rpoD, clpB, clpP, uspA, katG, uvrA, frdA, sodA, sodB, soi-28, narG, recA, xthA, his, lac, phoA, glnA and fabA.

The term “responding stress gene” refers to any gene whose transcription is induced as a result of environmental stress or by the presence of an environmental insult acting on a globally regulated stress circuit. “Responding stress genes” are under the control of globally regulated stress genes (Table 1) which are in turn responsive to environmental stresses. Typical E. coli stress genes encode stress proteins and include, but are not limited to groEL, groES, dnak, dnaj, grpE, lon, lysU, rpoD, clpB, clpP, uspA, katG, uvrA, frdA, sodA, sodB, soi-28, narG, recA, xthA, his, lac, phoA, ginA, micF, and fabA.

The term “globally regulated stress responsive gene” refers to genes that control a global genetic circuit that is responsive to environmental stresses. Typical enteric globally regulated stress responsive genes are listed in Table 1.

The term “oxidative stress responsive gene” refers to a gene responsive to any form of oxidative stress. Typical oxidative stress conditions responsible for up-regulating these genes are produced by reactions producing peroxides and superoxides.

The term “homolog” as applied to a gene means any gene derived from the same or a different microbe having the same function. Homologs additionally may a have significant sequence similarity.

The term “oxyR” refers to a gene characterized by the activation of its protein product by the presence of peroxides.

The term “soxR” refers to a gene characterized by the activation of its protein product by the presence of superoxides.

The term “soxs” refers to a gene characterized by up-regulation by SoxR.

The phrase “metabolic enzymatic pathway of interest” refers to an enzymatic pathway present in a microbial production host capable of producing an end product. Typically the metabolic enzymatic pathway will catalyze a series of reactions that will result in the formation of a chemical end product. In the alternative the end product may be an expressed protein. The “pathway” may comprise a number genes encoding a series of interacting proteins or may comprise a single gene producing a specific gene product. Metabolic enzymatic pathways may be introduced into a microbial production host, may be entirely native to the host or may be a chimera of foreign and native genes.

The term “chemical substance” refers to any substance with which the microbial host may come into contact that results in impairment of the host cells metabolic efficiency. Chemical substances are most often associated with specific fermentation reactions and conditions and may include organic solvents for example.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. “Inducible promoter” means any promoter that is responsive to a particular stimulus.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

The present invention provides a method for increasing the metabolic efficiency of a microbial production host expressing an enzymatic metabolic pathway. The method involves up-regulating globally regulated stress responsive genes in the host cell. Up-regulation of these genes has been shown to increase metabolic efficiency of native and foreign enzymatic pathways, increased expression of recombinant proteins, increased growth rate as well as increased resistance to substance toxicity.

Host Cells Harboring Highly Expressed Globally Regulated Stress Genes

One aspect of the present invention is a microbial host cell harboring a globally regulated stress gene that is highly expressed. The globally regulated stress gene may be native to the microbial host cell or may be introduced to the host cell using recombinant methods. Virtually any microbial host cell having an endogenous response to stress is suitable where bacteria, yeasts and fungi are preferred.

Globally regulated stress genes are known in the art and have been well documented. A listing of some the most fully characterized genes, as well as their regulating circuits is given in Table 1 below.

TABLE 1

| | REGU- | REGU- | |
|---|---|---|---|
| | LATORY | LATORY | RESPONDING |
| STIMULUS | GENE(S) | CIRCUIT | GENES* |
| Protein | rpoH | Heat Shock | grpE, dnaK, |
| Damage^{a} | | | lon, rpoD, groESL, |
| | | | lysU, htpE, htpG, |
| | | | htpl, htpK, clpP, |
| | | | clpB, htpN, htpO, |
| | | | htpX, etc. |
| DNA Damage^{b} | lexA, recA | SOS | recA, uvrA, |
| | | | lexA, umuDC, uvrA, |
| | | | uvrB, uvrC, sulA |
| | | | recN, uvrD, ruv, |
| | | | dinA, dinB, dinD, |
| | | | dinF etc. |
| Oxidative | oxyR | Hydrogen | katG, ahp, etc. |
| Damage^{c} | | Peroxide | |
| Oxidative | soxRS | Superoxide | micF, sodA, |
| Damaged | | | nfo, zwf, soi, etc. |
| Membrane | fadR | Fatty Acid | fabA |
| Damage^{e} | | Starvation | |
| Any^{f} | ? | Universal | uspA |
| | | Stress | |
| Stationary | rpoS | Resting State | xthA, katE, |
| Phase^{g} | | | appA, mcc, bolA, |
| | | | osmB, treA, otsAB, |
| | | | cyxAB, glgS, dps, |
| | | | csg, etc. |
| Amino Acid | relA, spoT | Stringent | his, ilvBN, |
| Starvation^{h} | | | ilvGMED, thrABC, |
| | | | etc. |
| Carbon | cya, crp | Catabolite | lac, mal, gal, |
| Starvationⁱ | | Activation | ara, tna, dsd, hut, |
| | | | etc. |
| Phosphate | phoB, phoM, | P Utilization | phoA, phoBR, |
| Starvation^{j} | phoR, phoU | | phoE, phoS, aphA, |
| | | | himA, pepN, ugpAB, |
| | | | psiD, psiE, psiF, |
| | | | psiK, psiG, psiI, |
| | | | psiJ, psiN, psiR, |
| | | | psiH, phiL, phiO, |
| | | | etc. |
| Nitrogen | glnB, glnD, | N Utilization | glnA, hut, etc. |
| Starvation^{k} | glnG, glnL | | |

*Genes whose expression is increased by the corresponding stimulus and whose expression is controlled by the corresponding regulatory gene(s).
^{a}Neidhardt and van Bogelen in E. coli and Salmonella typhimurium; Cellular and Molecular Biology (Neidhardt, F. C., et al. Eds., pp. 1334-1345, American Society of Microbiology, Washington, DC (1987))
^{b}Walker in E. coli and Salmonella typhimurium; Cellular and Molecular Biology (Neidhardt, F. C., et al. Eds., pp. 1346-1357, American Society of Microbiology, Washington, DC (1987))
^{c}Christman et al. Cell 41: 753-762 (1985); Storz et al. Science 248: 189-194 (1990); Demple, Ann. Rev. Genet. 25: 315-337 (1991)
^{d}Demple, Ann. Rev. Genet. 25: 31 337 (1991)
^{e}Magnuson et al. Microbiol. Rev 57: 522-542 (1993)
^{f}Nystrom and Neidhardt, J. Bacteriol, 175: 2949-2956 (1993); Nystrom and Neidhardt (Mol. Microbiol. 6: 3187-3198 (1992)
^{g}Kolter et al. Ann. Rev. Microbiol. 47: 855-874 (1993)
^{h}Cashel and Rudd in E. coli and Salmonella typhimurium; Cellular and Molecular Biology (Neidhardt, F. C., et al. Eds., pp. 1410-1438, American Society of Microbiology, Washington, DC (1987)); Winkler in E. coli and Salmonella typhimurium; Cellular and Molecular Biology (Neidhardt, F. C., et al. Eds., pp. 395-411, American Society of Microbiology, Washington, DC (1987))
ⁱNeidhardt, Ingraham and Schaecter. Physiology of the Bacterial Cell: A Molecular Approach, Sinauer Associates, Sunderland, MA (1990), pp 351-388; Magasanik and Neidhardt in E. coli and Salmonella typhimurium; Cellular and Molecular Biology (Neidhardt, F. C., et al. Eds., pp. 1318-1325, American Society of Microbiology, Washington, DC (1987))
^{j}Wanner in E. coli and Salmonella typhimurium; Cellular and Molecular Biology (Neidhardt, F. C., et al. Eds., E. coli and Salmonella typhimurium; Cellular and Molecular Biology (Neidhardt, F. C., et al. Eds., pp. 1326-1333, American Society of Microbiology, Washington, DC (1987))
^{k}Rietzer and Magasanik in E. coli and Salmonella typhimurium; Cellular and Molecular Biology (Neidhardt, F. C., et al. Eds., pp. 1302-1320, American Society of Microbiology, Washington, DC (1987)); Neidhardt, Ingraham and Schaecter. Physiology of the Bacterial Cell: A Molecular Approach, Sinauer Associates, Sunderland, MA (1990), pp 351-388

Thus, any microbial cell comprising the global regulatory stress circuits listed above are suitable for use in the present invention.

Of particular interest for use in the present invention are transcriptional regulators responsive to oxidative stress such as the OxyR and SoxRS proteins. Species of bacteria with demonstrated oxidative stress responses are particularly suitable and include, but are not limited to Mycobacterium, Brucella, Bacteroides, Erwinia, Streptomyces, Acinetobacter, Escherichia, Xanthomonas Pseudomonas, Chromobacterium, Arthrobacter, and Salmonella.

Most, if not all of the enteric bacteria have been shown to harbor an oxidative stress regulatory system and members of the enteric bacterial family are particularly suitable hosts. Enteric bacteria are members of the family Enterobacteriaceae, and include such members as Escherichia, Salmonella, and Shigella. They are gram-negative straight rods, 0.3-1.0×1.0-6.0 μm, motile by peritrichous flagella, except for Tatumella, or nonmotile. They grow in the presence and absence of oxygen and grow well on peptone, meat extract, and (usually) MacConkey's media. Some grow on D-glucose as the sole source of carbon, whereas others require vitamins and/or mineral(s). They are chemoorganotrophic with respiratory and fermentative metabolism but are not halophilic. Acid and often visible gas is produced during fermentation of D-glucose, other carbohydrates, and polyhydroxyl alcohols. They are oxidase negative and, with the exception of Shigella dysenteriae 0 group 1 and Xenorhabdus nematophilus, catalase positive. Nitrate is reduced to nitrite except by some strains of Erwinia and Yersina. The G+C content of DNA is 38-60 mol % (Tₘ, Bd). DNAs from species from species within most genera are at least 20% related to one another and to Escherichia coli, the type species of the family. Notable exceptions are species of Yersina, Proteus, Providenica, Hafnia and Edwardsiella, whose DNAs are 10-20% related to those of species from other genera. Except for Erwinia chrysanthemi all species tested contain the enterobacterial common antigen (Bergy's Manual of Systematic Bacteriology, D. H. Bergy, et al., Baltimore: Williams and Wilkins, 1984).

Up-Regulation of Stress Genes

Another aspect of the present invention is the up-regulation of genes involved in the stress response, and particularly the up-regulation of the regulatory genes. So for example, globally regulated stress genes of particular interest include but are not limited to rpoH, fadR, relA, spoT, cya, crp, phoM, phoR, phoU, glnB, glnD, glnG, glnL, oxyR, soxRS, rpoS, lexA and recA.

Those genes that control the global response to oxidative stress, such as the oxyR and soxRS genes are particularly useful in the present invention. The oxyR circuit is generally responsive to the presence of peroxides while the soxRS circuit is responsive to superoxides.

Generally regulons such as oxyR and sox RS may be negatively or positively controlled. In the case of positively controlled (activation) systems, high level constitutive alleles locking the activator in the ON (vs. the inactive) state can be selected. An example of high level, constitutive expression of positively controlled system is given by crp* alleles in which the CAP acitvator protein acts independently of its normal co-activator cAMP. The second way to achieve constitutive expression of positively controlled system is the placement of the regularory gene on a high copy plasmid, which will increase the titer of the regulatory protein within the cell. This approach works for systems in which activated form of the regulator is proportional to the total regulatory protein concentration.

For positively activated OxyR and SoxRS systems constitutive mutant allells have been selected for both oxyR and soxR genes which cause high level constitutive expression of the genes that they control. Placing oxyR in a multicopy plasmid does not increase target gene expression because essentially all of the OxyR protein produced in vivo is in the OFF state when cells are growing exponentially. In contrast, overproduction of SoxS increases SoxRS regulon expression because the SoxS protein directly activates its target genes.

A number of oxyR and soxRS homologs have been identified and all would be suitable for use in the present invention depending on the choice of microbial production host. For example oxyR homologs are known to be present in Escherichia coli [gi|2367332|gb|AE000470.1|AE000470[2367332]; Streptomyces coelicolor [gi|6759556|emb|AL137187.1|SC7A8[6759556]; Pasteurella multocida [gi|12721704|gb|AE006172.1|AE006172[12721704]; Mycobacterium leprae [gi|13093618|emb|AL583924.1|MLEPRTN8[13093618]; Brucella abortus [gi|4098964|gb|U81286.1|BAU81286[4098964]; Bacteroides fragilis [gi|9944340|gb|AF206034.1|AF206034[9944340]; Erwinia chrysanthemi [gi|4583558|emb|AJ005255.1|ECAJ5255[4583558]; Haemophilus influenzae [gi|6626252|gb|L42023.1|L42023[6626252]; Acinetobacter gi|2462044|emb|Z46863.1 ACRBDOXN[2462044]; Xanthomonas campestris [gi|2098745|gb|U94336.1|XCU94336[2098745]; and Bacillus subtilis [gi|1805369|dbj|D50453.1|D50453[1805369].

Similarly soxRS homologs are known to be present in Escherichia coli [gi|2367340|gb|AE000479.1|AE000479[2367340]; Erwinia chrysanthemi [gi|11342544|emb|AJ301654.1|ECH301654[11342544]; Streptomyces coelicolor [gi|1228443|emb|AL450165.1|SC5F1[11228443]; Vibrio cholerae [gi|9657462|gb|AE004351.1|AE004351[9657462]; Pseudomonas aeruginosa [gi|6715613|gb|U19797.2|PAU19797[6715613];

Chromobacterium violaceum [gi|3820506|gb|AF061445.1|AF061445[3820506]; Mycobacterium tuberculosis [gi|3261610|emb|Z77163.1|MTCY339[3261610]; Arthrobacter [gi|3116219|dbj|AB007122.1|AB007122[3116219]; and Salmonella typhimurium[gi|1421770|gb|U61147.1|STU61147[1421770].

It is an aspect of the invention that globally regulated stress genes be up-regulated in order to see an increase in the metabolic efficiency of the cell and its expressed pathways. Up-regulation may be a result of constitutive expression, or as a result of induction or over-expression of the genes. It is possible to create constitutively expressed stress genes by a variety of means, the most common however is by random or site specific mutagenesis on regulatory genes.

Methods of creating mutants are common and well known in the art. For example, wild type cells containing the globally regulated genes may be exposed to a variety of agents such as radiation or chemical mutagens and then screened for the desired phenotype. When creating mutations through radiation either ultraviolet (UV) or ionizing radiation may be used. Suitable short wave UV wavelengths for genetic mutations will fall within the range of 200 nm to 300 nm where 254 nm is preferred. UV radiation in this wavelength principally causes changes within nucleic acid sequence from guanidine and cytosine to adenine and thymidine. Since all cells have DNA repair mechanisms that would repair most UV induced mutations, agents such as caffeine and other inhibitors may be added to interrupt the repair process and maximize the number of effective mutations. Long wave UV mutations using light in the 300 nm to 400 nm range are also possible but are generally not as effective as the short wave UV light unless used in conjunction with various activators such as psoralen dyes that interact with the DNA.

Mutagenesis with chemical agents is also effective for generating mutants and commonly used substances include chemicals that affect nonreplicating DNA such as HNO₂ and NH₂OH, as well as agents that affect replicating DNA such as acridine dyes, notable for causing frameshift mutations. Specific methods for creating mutants using radiation or chemical agents are well documented in the art. See for example Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

After mutagenesis has occurred, mutants having the desired phenotype may be selected by a variety of methods. Random screening is most common where the mutagenized cells are selected for the ability to produce the desired product or intermediate. Alternatively, selective isolation of mutants can be performed by growing a mutagenized population on selective media where only resistant colonies can develop. Methods of mutant selection are highly developed and well known in the art of industrial microbiology. See Brock, Supra., DeMancilha et al., Food Chem., 14:313, (1984).

Within the context of the present invention, oxyR and soxR mutants have been generated and isolated that constitutively express the OxyR and SoxRS regulated genes. These mutants are well characterized in the literature. (see for example Morgan et al., Proc. Natl. Acad. Sci. U.S. A.,83(21):8059-63,(1986); Christman et al., Cell. 41(3):753-62, (1985); and Christman et al., Proc Natl Acad Sci USA., 86(10):3484-8, (1989) for oxyR mutants, and Greenberg et al. Proc Natl Acad Sci USA., 87(16):6181-5, (1990); Hidalgo et al., Cell, 88(1):121-9, (1997), for soxR mutants).

Alternatively it may be useful to generate genetic chimera where inducible promoters are operably linked to native or foreign globally regulated stress genes. This will allow for the regulation of the increased stress response at a time in the production process when it is most advantageous. Similarly, it may be useful to introduce multiple copies of foreign or native stress genes into a host for the up-regulation of the stress response.

The creation of chimeric genes is common and well known in the art. For reasons of convenience, the chimeric genes may comprise promoter sequences and translation leader sequences derived from the same genes. 3′ Non-coding sequences encoding transcription termination signals must also be provided. Any combination of any promoter and any terminator capable of inducing expression of a coding region may be used in the chimeric genetic sequence. Some suitable inducible promoters useful in enteric bacteria include but are not limited to tac; lac; araBAD; lambda pL and gal.

Enhanced Metabolic Efficiency

In a preferred embodiment a host cell comprising an up-regulated stress response is used to enhance the metabolic efficiency of the cell. Enhanced metabolic efficiency is manifested in a variety of ways including increased metabolic efficiency of native and foreign enzymatic pathways, increased expression of recombinant proteins, increased growth rate as well as increased resistance to substance toxicity.

Metabolic Enzymatic Pathways

Metabolic enzymatic pathways of the present invention may be a series of genes encoding proteins of interacting functions that result in the generation of a specific product, or they may include a single gene expression single protein gene product. One metabolic enzymatic pathway of interest that will benefit from the present invention is the pathways that results in the production of 1,3-propanediol. 1,3-Propanediol is a monomer having potential utility in the production of polyester fibers and the manufacture of polyurethanes. Recently a single metabolic pathway has been engineered in a single host cell for the production of this monomer. The details of the construction of this pathway are detailed in U.S. Pat. No. 5,686,276; U.S. Pat. No. 6,025,184; and U.S. Pat. No. 5,633,362, the disclosure of which is hereby incorporated by reference.

Briefly, the relevant enzymatic pathway comprises a series of enzymes that will convert glucose to 1,3-propanediol. First glucose is converted in a series of steps by enzymes of the glycolytic pathway to dihydroxyacetone phosphate (DHAP) and 3-phosphoglyceraldehyde (3-PG). Glycerol is then formed by either hydrolysis of DHAP to dihydroxyacetone (DHA) followed by reduction, or reduction of DHAP to glycerol 3-phosphate (G3P) followed by hydrolysis. The hydrolysis step can be catalyzed by any number of cellular phosphatases which are known to be non-specific with respect to their substrates or the activity can be introduced into the host by recombination. The reduction step can be catalyzed by a NAD⁺ (or NADP⁺) linked host enzyme or the activity can be introduced into the host by recombination. It is notable that the dha regulon contains a glycerol dehydrogenase (E.C. 1.1.1.6) which catalyzes the reversible reaction of Equation 3.

Glycerol→3-HP+H₂O (Equation 1)

3-HP+NADH+H⁺→1,3-Propanediol+NAD⁺ (Equation 2)

Glycerol+NAD⁺→DHA+NADH+H⁺ (Equation 3)

Glycerol is converted to 1,3-propanediol via the intermediate 3-hydroxy-propionaldehye (3-HP) as has been described in detail above. The intermediate 3-HP is produced from glycerol, Equation 1, by a dehydratase enzyme which can be encoded by the host or can introduced into the host by recombination. This dehydratase can be glycerol dehydratase (E.C. 4.2.1.30), diol dehydratase (E.C. 4.2.1.28) or any other enzyme able to catalyze this transformation. Glycerol dehydratase, but not diol dehydratase, is encoded by the dha regulon. 1,3-Propanediol is produced from 3-HP, Equation 2, by a NAD⁺- (or NADP⁺-) linked host enzyme or the activity can introduced into the host by recombination. This final reaction in the production of 1,3-propanediol can be catalyzed by 1,3-propanediol dehydrogenase (E.C. 1.1.1.202) or other alcohol dehydrogenases.

The genes encoding the enzymes of the 1,3-propanediol pathway are known in the art and may be assembled from a variety of sources and used to transform a number of suitable hosts. Up-regulation of the stress responsive genes and particularly the oxidative stress genes in these hosts will result in the enhanced efficiency of the 1,3-propanediol pathway, producing greater yields of end product.

Over Expression of Protein

Up-regulation of the stress responsive genes of the host cell will also have the effect of increasing the yield of a recombinantly expressed protein. The use of recombinant systems to produce commercially useful proteins is common. A non-limiting list of recombinantly produced proteins includes for example those of medical and pharmaceutical interest such as collagen, human lactoferrin, human ribonuclease, beta-interferon, human growth, and recombinant Protein A, as well as a large number of industrial enzymes.

Host cells comprising a stress responsive gene or genes that may be up-regulated can be engineered to incorporate the genetic machinery necessary for the expression of recombinant proteins. Vectors or cassettes useful for the transformation of suitable host cells with recombinant genes are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host.

Initiation control regions or promoters, which are useful to drive expression of the instant ORF's in the desired host cell are numerous and familiar to those skilled in the art. Virtually any promoter capable of driving these genes is suitable for the present invention including but not limited to CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, lP_{L}, lP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

Once the host comprising both the recombinant gene to be expressed and the up-regulated stress gene is constructed it may be used for enhanced production of the recombinant protein.

Resistance to Chemical Substances

Another aspect of the present invention is the increased resistance of the host cell harboring an up-regulated stress responsive gene to various toxic organics. In some industrial fermentation methods it is necessary to bring cells in contact with various solvents or toxic carbon sources. For example, where there is an interest in converting toluene to pHBA (PCT/US98/12072) cells are exposed to a high level of toluene for the conversion. One of the limiting factors in this process is the cells ability to withstand the toxic effects of toluene. Cells of the present invention harboring an up-regulated stress responsive gene will have a greater tolerance to these toxic organics than those without this feature. By toxic organics it is mean any carbon containing material that may be used by the cell as a carbon source but is toxic to that cell. Typical toxic organics include but are not limited to toluene, xylene, 1,2,3,4-tetrahydronaphthalene (tetralin), benzene, cyclohexane, and alcohols such as cyclohexanol, and methanol.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of bacterial cells were obtained from DIFCO Laboratories (Detroit, Mich.), GIBCO BRL® Life Technologies(Rockville, Md.), or Sigma-Aldrich Chemical Company (St. Louis, Mo.) unless otherwise specified.

The meaning of abbreviations is as follows: “h” means hour(s), “min” means minute(s), “sec! means second(s), “d” means day(s), “mL” means milliliters, “L” means liters.

The meaning of abbreviations is as follows: “h” means hour(s), min” means minute(s), “sec” means second(s), “d” means day(s), “mL” means milliliters, “L” means liters.

### Example 1

### Construction of Constitutive oxyR (oxyR2) Strains

E. coli stain TA4110 (Christman et al., Cell, 41: 753-762(1985)) was used as the donor for an oxyR constitutive mutant allele A233V (also known as oxyR2) (Christman et al., Proc. Natl. Acad. Sci. USA, 86: 3484-3488 (1989)). P1 transduction was used to move the oxyR2 allele into recipient strains MC4100 or BL21(DE3)pLysS (Invitrogen, Carlbad, Calif.) according to procedures described in Miller, J., (A Short Course in Bacterial Genetics, Cold Spring Harbor, N.Y., Cold Spring Harbor Laboratory, (1992)). Briefly, bacterial phage P1 was grown in 10 mL of TA4110 until cells were completely lysed. After removal of cell debris by centrifugation, 100 μL of the P1 lysate was used to infect 200 μL recipient strain grown to OD₆₀₀˜1 in LB medium. Then, oxyR2 mutants were selected as peroxide resistant colonies on minimum glucose plate containing 1 mM hydrogen peroxide. The minimum glucose plate was made according to recipe in Miller (supra). Colonies picked were further checked with zone of inhibition assays with hydrogen peroxide and cumene hydroperoxide to make sure that they were resistant to both types of peroxides. Briefly, cells were grown overnight at 37° C. in LB medium prior to testing. Aliquots (0.1 mL) of cultures were then added to soft agar and were plated on LB plates. Ten microliters of either 30% hydrogen peroxide or cumene hydroperoxide were applied to a ¼ in diameter filter disc (Becton Dickinson, Cockeysville, Md.) placed in the center of the agar. The diameter of the zone of killing was measured after 24 h at 37° C. Typical numbers were listed in Table 1.

TABLE 1
Diameter of the zone killed

| | wild type | oxyR2 |
|---|---|---|
| Hydrogen peroxide | 25 mm | 13 mm |
| Cumene hydroperxide | 26 mm | 14 mm |

### Example 2

### Construction of pRSETB-ECFP Plasimd

The protein over-expression vector pRSETB was obtained from Invitrogen (Carlsbad, Calif.), and plasmid pECFP-1 containing ECFP gene was obtained from Clontech (Palo Alto, Calif.). The ECFP gene was PCR amplified using pECFP-1 as template, with two primers (5′-GAT AAG GAT CCG ATG GTG AGC MG-3′ (SEQ ID NO:1) and 5′-TTC GM TTC CTT GTA CAG CTC GTC-3′ (SEQ ID NO:2)) that incorporate BamHI and EcoRI sites in the 5′ and 3′ end of the ECFP gene, respectively. After restriction digestion with EcoRI and BamH1, the PCR product was purified with a Qiagen PCR purification kit (Qiagen, Valencia, Calif.), and ligated to the EcoRI/BamHI site of the pRSETB vector. The ligation product was used to transform competent E. coli DH5α cells (GIBCO BRL® Life Technologies) to AmpR (transformants selected by ampicillin resistance). The plasmid was then isolated from the selected transformants and the insert was verified by restriction digestion with EcoRI and BamHI and by direct sequencing.

### Example 3

### Protein Over-Expression

Strain BL21(DE3)pLysS/oxyR⁺ (Invitrogen) and BL21(DE3)pLysS/oxyR2 (made as described previously) were chosen as the protein production hosts. BL21 (DE3)pLysS is a commercial E. coli strain that has bacterial phage T7 RNA polymerase integrated into the E. coli chromosome as lysogen and a pLsyS plasmid that contains T7 lysozyme to minimize basal level protein expression. The plasmid pRSETB-ECFP was used to transform BL21(DE3)pLysS/oxyR⁺ and BL21 (DE3)pLysS/oxyR2. Transformants were selected by their ability to grow in the presence of ampicillin. A protein over-expression experiment was carried out according to procedures described in the Users Manual for BL21 (DE3)pLysS (Invitrogen) to verify that the systems were capable of over expressing the ECFP protein. Briefly, a 5 mL LB medium was inoculated with a single colony and allowed to grow at 37° C. to OD₆₀₀˜0.8. This first culture was diluted 1:20 into 10 mL LB medium. The cultured cells were allowed to grow at 37° C. When the second culture reached OD₆₀₀˜0.6, it was induced with 1 mM IPTG and cultured for additional 2 to 3 h. Protein gels were run on cell pellet to visualize protein induction.

### Example 4

### Growth Comparison Between E. coli Wild Type and oxyR2Mutants

Single colonies of the wild type (MC4100/oxyR⁺) and its oxyR2 mutant (MC4100/oxyR2) derivative were picked from plates and used to inoculate two 5 mL aliquots of LB medium. Cells were grown in a 37° C. shaker (Environ Shaker, Lab-Line Instruments, Inc, Melrose Park, Ill.) with a shaking rate of 275 rpm. After overnight culturing, optical densities at 600 nm of the cells were measured and recorded in Table 2.

TABLE 2
Growth Comparison of Over-night Culture

Strains

| | MC4100/oxyR⁺ | MC4100/oxyR2 |
|---|---|---|
| OD at 600 nm | 1.89 | 2.23 |

Data represents average of three independent experiments.

This experiment showed that the oxyR2 mutant could produce more cell mass than the wild type stain under same growth conditions. This could be a very useful trait for material productions in which the final product is proportional to total cell mass.

### Example 5

### Resistance to Organic Solvent

Wild type MC4100/oxyR⁺ and MC4100oxyR2 mutants were streaked onto the same LB plate made with a 80 mm diameter PYREX Petri dish (VWR International, West Chester, Pa.). Solvent tetralin (1,2,3,4-tetrahydronaphthalene) was then poured onto the LB agar surface to have a 5 mm depth of overlay. The plate was then covered with a Petri dish cover and sealed with a strip of parafilm (American National Can, Chicago, Ill.), and put into 37° C. incubator. After overnight culturing, tetralin was decanted and the plate was put into a fume hood for 30 min to allow evaporation of the remaining tetralin. A picture of the plate was taken with an Eagle Eye® II still imaging system (Stratagene, La Jolla, Calif.). No colony was observed on the left side of the plate where the wild type strain (oxyR⁺) had been streaked, whereas the oxyR2 mutant strain on the right side of the plate grew just fine.

This experiment showed that the oxyR2 mutant is resistant to organic solvent tetralin. The mechanism of tetralin toxicity has been studied by Ferrante et al., (Proc Natl Acad Sci USA. 92:7617-21, (1995)). They proposed that the toxicity is not from tetralin itself, but rather from its peroxide derivative formed through auto-oxidation. They showed that the E. coli enzyme alkyl hydroperoxide reductase (Ahp) eliminates toxicity by reducing the peroxide to alcohol. Ahp is regulated by OxyR, and it is overexpressed in the oxyR2 mutant. This is probably why the oxyR2 mutant confers resistance to tetralin. Since auto-oxidation is a common in organic compounds and it is a general phenomenon, we propose that the oxyR2 mutant may be useful for a wide spectrum of organic solvents.

Example 6

### Plasmid Stability

Single colonies of the wild type BL21 (DE3)pLysS/pRSETB-eCFP and its oxyR2 mutant derivative were picked from plates and used to inoculate two 5 mL aliquots of LB medium containing 50 mg/L ampicillin. Cells were grown in a 37° C. shaker with a shaking rate of 275 rpm. After overnight culturing, cells were diluted and plated on LB plates with 50 mg/L ampicillin. The number of colonies on each plate was counted manually and recorded in Table 3.

TABLE 3
Colony count for plasmid stability

| | Strains | |
|---|---|---|
| | BL21(DE3)pLysS/oxyR⁺ | BL21(DE3)pLysS/oxyR2 |
| Colonies (x100,000) | 12 | 835 |

Data represents average of two independent experiments.

This example was used to demonstrate the utility of the oxyR2 mutant in production of toxic material in E. coli. It has been found that green fluorescence protein (GFP) and its derivatives are toxic when overexpressed inside cells, probably because the GFP chromophore promotes formation of reactive oxygen species (Haseloff and Amos, Trends Genet, (8):328-9 (1995); Liuet al. Biochem Biophys Res Commun., 260:712-7, (1999)). The experiment described above showed that the ECFP expression plasmid was not stable in the production strain BL21 (DE3)pLysS/oxyR⁺. Overnight culturing resulted in the loss of the plasmid by a large portion of cells. Loss of the expression plasmid due to instability would create an unacceptable inefficiency in an industrial production process. When the oxyR mutation (oxyR2) was moved into the production strain BL21(DE3)pLysS, the stability of the plasmid was greatly improved. The number of colonies from the stress-resistant strain was 69 times higher than that from the wild type strain.

What is claimed is:

1. A method for increasing the metabolic efficiency of a microbial enzymatic pathway of interest comprising:
a) providing a microorganism having:
i) at least one highly expressed globally regulated stress responsive gene
ii) a metabolic enzymatic pathway of interest; and
b) growing the microorganism of (a) for a time sufficient to produce an end-product from the microbial enzymatic pathway of interest

2. A method according to claim 1 wherein the metabolic efficiency is increased as a result of increased resistance to a chemical substance.

3. A method according to claim 2 wherein the chemical substance is a toxic organic molecule.

4. A method according to claim 3 wherein the toxic organic molecule is selected from the group consisting of xylene, 1,2,3,4-tetrahydronaphthalene (tetralin), benzene, cyclohexane, and alcohols.

5. A method according to claim 2 wherein the chemical substance is Green Fluorescent Protein.

6. A method according to claim 1 wherein the microorganism is selected from the group consisting of bacteria, yeast and fungi.

7. A method according to claim 6 wherein the microorganism is an enteric bacteria.

8. A method according to claim 6 wherein the microorganism is selected from the group consisting of Mycobacterium, Brucella, Bacteroides, Erwinia, Streptomyces, Acinetobacter, Escherichia, Xanthomonas Pseudomonas, Chromobacterium, Arthrobacter, and Salmonella.

9. A method according to claim 8 wherein the enteric bacteria is E. coli.

10. A method according to claim 1 wherein the metabolic enzymatic pathway of interest is native to the microorganism.

11. A method according to claim 1 wherein the metabolic enzymatic pathway of interest is foreign to the microorganism.

12. A method according claim 1 wherein the end product of the metabolic enzymatic pathway of interest is a protein.

13. A method according to claim 12 wherein the end product of the metabolic enzymatic pathway of interest is 1,3-propanediol.

14. A method according to claim 1 wherein the globally regulated stress responsive gene is selected from the group consisting of rpoH, fadR, relA, spot, cya, crp, phoM, phoR, phoU, glnB, glnD, glnG, glnL, oxyR, soxRS, rpoS, lexA and recA.

15. A method according to claim 1 wherein the stress responsive gene is a globally regulated oxidative stress gene which is responsive either to the presence of peroxides or superoxides.

16. A method according to claim 15 wherein the oxidative stress gene is selected from the group consisting of oxyR and soxRS and homologs thereof.

17. A method according to claim 1 wherein the stress responsive gene is constitutively expressed.

18. A method according to claim 1 wherein the stress responsive gene is inducibly regulated.
